(19) United States
(12) Patent Application Publication
(10) Pub. No.: US 2009/0124825 A1
(43) Pub. Date: May 14, 2009

(54) Method of (meth) acrylate production

(75) Inventors: Yong Tang, Shanghai (CN); Chunlei Zhang, Shanghai (CN); Jianxue Ma, Shanghai (CN); Jingming Shao, Shanghai (CN)

Correspondence Address: DAVID AND RAYMOND PATENT FIRM, 108 N. YNEZ AVE., SUITE 128, MONTEREY PARK, CA, 91754, US

(73) Assignee: Shanghai Huayi Acrylic Acid Co., Ltd

(21) Appl. No.: 11/985,409

(22) Filed: Nov. 14, 2007

(51) Int. Cl. C07C 69/52

(52) U.S. Cl. 560/205

(57) ABSTRACT

A method of methacrylate production includes the steps of providing methacrylate gas mixture; absorbing said gas mixture by water to form a solution mixture; introducing said solution mixture into a distillation column, separating impure methacrylate from said solution mixture in said distillation column that solution of said impure methacrylate is collected at said stripping section of said distillation column; and removing acetic acid from said impure methacrylate to produce methacrylate. It effectively enhances the separation capacity of the distillation column, lowers the whole column pressure drop and operation temperature, avoids the polymerization tendency of the methacrylate under high temperature, to make the water content of the bottom discharging decreases to 0.6%, acetic acid content reduces to below 2%, methyl acrylic content of the top column water phase decreases to 0.5%, thereby reduces the methacrylate products unit consumption and improves the product quality.

### BACKGROUND OF THE PRESENT INVENTION

1. Field of Invention

The present invention relates to a method of (meth)acrylate production, and particularly to a new distillation column for separating (meth)acrylate from (meth) acrylate solution, wherein the rectification section of the distillation column is using structured packing plate in the upper section, and jet co-flow packing tray in the lower section, the jet co-flow packing trays take 20-80% of the total plates of the rectification section. The stripping section is using guide valve tray in the upper section, and vertical sieve tray in the lower section, the guide valve trays take 20-80% of the total plates of the stripping section.

2. Description of Related Arts

It is well known, the method of (meth)acrylate production comprises selecting at lest one compound from C₃ compounds such as propane, propylene, isopropyl alcohol, glycerol or acrolein, or from C4 compounds such as isobutane, isobutylene, tert-butanol or methylpropenal, as raw materials for catalytic oxidation of gas mixture containing (meth)acrylate. The gas mixture contains unreacted raw materials such as propane, propylene, isobutane and isobutylene, and the reaction by-product impurities such as (meth)acrolein, formic acid, acetic acid, formaldehyde, acetaldehyde, maleic acid, propionic acid, and furfural. The gas mixture containing (meth)acrylate with unreacted raw materials and by-product impurities is usually collected as (meth)acrylate solution by contacting with the absorption solvent, and the solvent is isolated through distillation, then the low boiling point and high boiling point components are selectively separated respectively. Ultimately, the (methyl)acrylate products are achieved.

There are two technologies in the production of acrylate by propylene oxidation: one technology utilizes two oxidation processes of oxidizing propylene to get acrolein and oxidizing acrolein to get acrylate which are performed in different reactors, and under different reaction conditions; the other technology utilizes one oxidation process which oxidizing propylene directly to get acrylate. For both technologies, the reactors are filled with gas mixture such as Mo group catalysts, acrylate containing gas mixture using propylene and humidifying air as feed gas through catalytic oxidation; the gas mixture contacts with water in the absorption column forming aqueous solution of acrylate; the aqueous solution of acrylate is then incorporated with appropriate azeotropic agent for azeotropic distillation in the distillation column, the azeotropic mixture of water, acetic acid and azeotropic agent is distilled from the top of the azeotropic distillation column, to remove water and most of acetic acid from the aqueous solution of acrylate, the acrylate is collected at the bottom of the distillation column.

Additionally, other technologies of acrylate production can also be utilized, such as using propane instead of propylene in the catalytic oxidation in the presence of Mo—V—Te group complex oxide catalyst or Mo—V—Te group complex oxide catalyst; and using isobutene or tert-butanol instead of propylene in the catalytic oxidation in the presence of Mo—Bi—Fe group complex oxide catalyst or Mo—V—P group complex oxide catalyst.

(Meth)acrylate and its ester is an unsaturated double bond compound, its chemical nature is very active, and the vinyl it contains is quite easy to self-polymerize or co-polymerize with other monomers. The primary concern in the design of a (meth)acrylate distillation system is to avoid the (meth)acrylate self-polymerization, because polymerization performs very quickly, and generates a lot of heat, once occurs it is very difficult to control. Temperature is the most common factor to active the polymerization reaction. The higher the temperature, the easier to active the polymerization reaction, and the more volume of polymers will be generated. Therefore, to control the production operation of the (meth)acrylate distillation at a low temperature is the most important factor.

(Meth)acrylate is very easy to polymerize. During the distillation process mentioned above, although adding inhibitor to aqueous Solution of (meth)acrylate can inhibit polymerization, it still contains (methyl)acrylate bi-polymers and other polymers in the solution. Since these polymers are generated in distillation operation, ordinary packed column is not suitable for the distillation process mentioned above. In the above technology of crude acrylate and crude methacrylate distillation, the distillation column is generally using non-weir large holes sieve tray structure. Because the (meth) acrylate polymerization occurs in the bottom of the column and the stripping section, using the large perforation sieve tray which is easy to clean-up is appropriate in this part. But still using large holes sieve tray in the rectification section which is not easy to polymerize will definitely affect the efficiency of the entire column, which lowers the capacity of the distillation column.

As the China patent CN1266050, it is using dehydrating column for aqueous solution of (meth)acrylate dehydration distillation. At least 3 ideal plates of distillation column are used as dehydration column. In detail, inside the column sieve tray, double flux plate, ripple plate or Intalox metal packing. In the bottom of the column, the temperature is 83-92° C., the acetic acid content is 2.3%, and the water content is 0.6%. As the China patent CN1477092, the total number of the plates in the acrylate distillation dehydration column is 21. The rectifying section of the column is using 11 large holes perforated sieve trays with the perforated rate of 10-20%. The upper section of the stripping section is using 5 jet co-flow packing trays. The acetic acid content at the bottom of the column is controlled below 2%. The acrylate consumption at the top of the column is below 1.5%. The whole column pressure drop of is kept in around 6 kPa. The temperature of the bottom of the column is controlled below 82° C. The number of ideal plates of the whole column is 6. While using large holes sieve tray structure in the stripping section is a compromise because the high concentration of the acrylate makes it easy to polymerize. It reduces the flux rate, tremendously constrains the whole column perform capability; at the same time, the pressure drop of the stripping section is relatively large. It is difficult to maintain the operation temperature of the column kettle in a lower rang, and can't fundamentally solve the problem of acrylate polymerization at the column bottom. As the China patent CN1745055A, the aqueous solution of acrylate distillation column is using 30 ripple plate non-weir perforated trays and flat plate non-weir perforated trays, arranged in such a manner that each group has 4 ripple plate non-weir perforated trays and 1 flat plate non-weir perforated tray, arranged upward. The perforated tray groups are arranged upward. The number of ideal plates is 9. The whole column pressure drop is 6.5 kPa. The temperature of the bottom of the column is controlled below 82° C. The acetic acid content at the column bottom is 2.3%, the water content is 0.6%. The operation cycle is 90 days.

### SUMMARY OF THE PRESENT INVENTION

To avoid the disadvantages of the inventions mentioned above, the present invention provides a new distillation column of separating (meth)acrylate from aqueous solution of (meth)acrylate. The rectification section of the distillation column is using structured packing tray and/or jet co-flow packing tray, the stripping section is using guided valve tray and/or vertical sieve tray. The structure of the distillation column effectively improved the separation capability, reduces the whole column pressure drop and operation temperature, thereby reduces the (meth)acrylate products unit consumption, improves the product quality, stably maintains the high (meth) acrylate recovery rate, and keeps the distillation column working continuously and stably in long term.

These and other objectives, features, and advantages of the present invention will become apparent from the following detailed description, the accompanying drawings, and the appended claims.

### BRIEF DESCRIPTION OF THE DRAWINGS

FIG. 1 is the flow diagram of the acrylate production, wherein M1 refers to mixer; R1 refers to first oxidation reactor; T1 refers to absorber; T2 refers to azeotropic distillation dehydrator; T3 refers to acetic acid stripper; T4 refers to acrylate product tower; T5 refers to cracking reactor; 1 refers to air; 2 refers to steam; 3 refers to propylene; 4 refers to oxidation reacting gas mixture; 5 refers to water; 6 refers to aqueous solution of acrylate; and 7 refers to acrylate products.

FIG. 2 illustrates the structural configuration of the vertical sieve tray.

FIG. 3 illustrates the structural configuration of the jet co-flow packing tray.

FIG. 4 is the perspective view of the guide valve tray.

FIG. 5 illustrates the structure of the distillation tower, wherein 1 refers to upper section of the rectification section (structured packing); 2 refers to lower section of the rectification section (jet co-flow packing tray); 3 refers to feed; 4 refers to upper section of the stripping section (guide valve tray); and 5 refers to lower section of the stripping section (vertical sieve tray).

### DETAILED DESCRIPTION OF THE PREFERRED EMBODIMENT

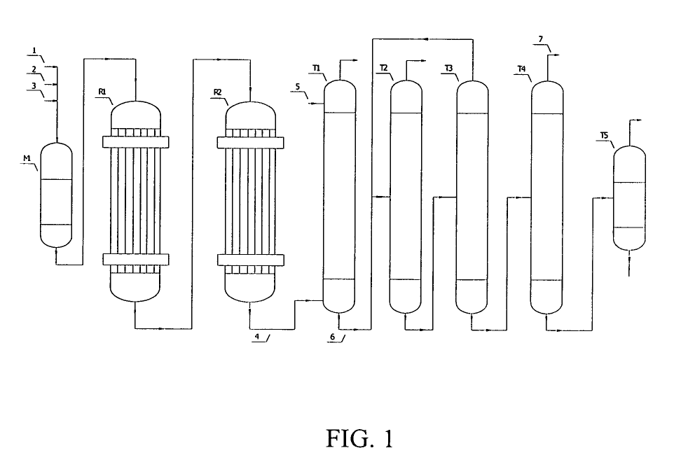

The present invention, with the premise of not damaging the effect of the present invention, includes all related methods and processes. Some of the ordinary processes of producing (meth)acrylate can be illustrated, referring to FIG. 1, an example of the acrylate is:

Propane, propylene or acrolein are mixed with humidified air in the mixer M 1, and performed the catalytic oxidation reaction in the first oxidation reactor R1 and the second oxidation reactor R2. The produced acrylate containing gas mixture enters into the absorber T1 which uses water or recycled water to absorb the acrylate containing gas mixture and generate aqueous solution of acrylate. Feed the aqueous solution of acrylate which contains acrylate, acetic acid and a small amount of formic acid and formaldehyde continuously into an azeotropic distillation dehydrator T2. Use azeotropic solvent such as toluene and halogenated aromatic hydrocarbons to distill the water, part of the acetic acid from the aqueous solution of acrylate, after being still stratified by the condensate in the top of the column, the toluene phase returns to the distillation column as reflux, the water phase is removed, and part of the water is used as absorbent in the acrylate reaction gas absorber; the remaining acetic acid is removed from the crude acrylate at the bottom of the column by the acetic acid stripper T3. The bottom liquid enters into acrylate product column T4. The acrylate product is received by refining separation. Feed the acrylate polymers and the inhibitor containing high boiling-point heavy components generated by other units as crude materials into the cracking reactor T5 for valuable products recovery.

The ordinary method of (meth)acrylate productions also comprises the operation units such as: oxidation unit for propane, propylene, or acrolein catalytic oxidation to generate acrylate containing gas mixture; absorption unit using water or recycled water to absorb acrylate containing gas mixture to generate aqueous solution of acrylate. An extraction unit contains an extraction solution such as toluene and acetic acid ester to extract acrylate from the aqueous solution of acrylate. A recovery unit is arranged for distillation of acrylate from extraction solution. A purification unit is arranged for distillation of acrylate products. A heavy components cracking unit uses acrylate polymers and the inhibitor containing high boiling-point heavy components generated by other units as crude materials into cracking reactor for valuable products recovery.

In addition, during the process of (meth)acrylate production, in order to inhibit the formation of (meth)acrylate polymers such as dimers, trimers, and four polymers, the inhibitor is commonly used. The inhibitor could be, for example, phenolic compounds, copper dithiocarbamate compounds, phenothiazine compounds, and copper acrylate. The copper dithiocarbamate compounds include: copper dialkyldithiocarbamate such as copper dimethyldithiocarbamate, copper diethyldithiocarbamate, copper dipropyldithiocarbamate, copper dibutyldithiocarbamate; cyclic copper alkylidenedithiocarbamate such as copper ethylidenedithiocarbamate, copper tetramethylenedithiocarbamate, copper pentamethylenedithiocarbamate, hexamethylenedithiocarbamate; cyclic copper oxydialkylenedithiocarbamate such as oxydiethylidenedithiocarbamate. The phenolic compounds include hydroquinone, methylhydroquine, pyrocatechol, resorcinol, phenol, cresol, or hydroquinone monomethyl ether, etc. Phenothiazine compounds include phenothiazine, dual-(α-methyl-benzyl) phenothiazine, 3,7-di-phenothiazine, dual-(α-dimethyl benzyl) phenothiazine, etc.

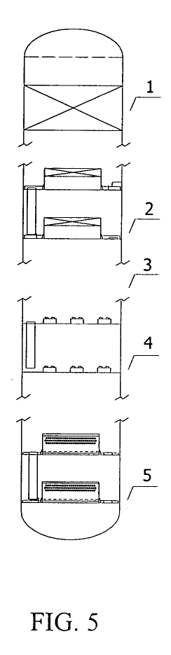

Referring to FIG. 5, in the distillation process, the rectification section of the distillation column is using structured packing tray in the upper section, and jet co-flow packing tray in the lower section. The stripping section is using guide valve tray in the upper section, and vertical sieve tray in the lower section. Because the different conditions such as the properties of the separation medium, the ratio of each type of column inner parts can be adjusted accordingly. The jet co-flow packing trays take 20-80% of the total plates of the rectification section, and the guide valve trays take 20-80% of the total plates of the stripping section.

The structured packing tray used in the upper section of the rectification section can be ripple ceramic packing, metal mesh packing, ceramic structured packing, or metal large-wave board, optimally using ripple ceramic packing. According to the moving characteristics of gas in structured flow channel, the packing has plural of pulse area, in the pulse area, the airflow raising direction changes from raising tilt upward to raising vertically, the resistance drops suddenly, then gradually the raising direction changes back to tilt upward, the resistance changing from large to small, and then form small to large generates a pulse movement of the vapor-liquid two-phase. The several vapor-liquid pulses in the packing can strengthen the vapor-liquid turbulence, significantly improve the separation efficiency, and reduce the resistance.

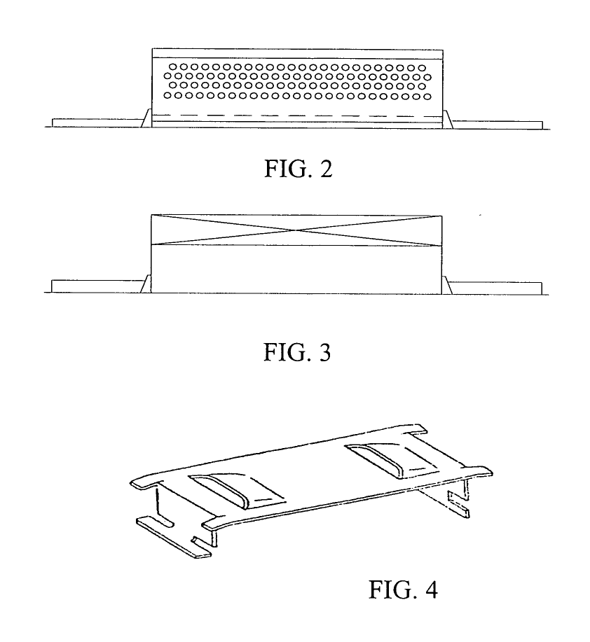

Referring to FIG. 2, the sieve tray has many structures. It has big holes (such as round, square, rectangular), over the holes are settled with various bubble caps correspondingly, and downcomer. On the ordinary trays, the vapor-liquid flow is in bubble state, and contacts in cross low. On the new trays, the vapor-liquid flow contact is in injection state as illustrated in FIG. 2. Liquid from the upper tray flows out of the downcomer, passes through the rows of bubble caps horizontally, flows into the bubble caps by the bottom gap. The vapor from the lower tray rises from the perforated tray and bubbles the liquid, the vapor flow and the liquid membrane exchanges the momentum in the bubble caps, the liquid bubble is broken into drops and foams. In the bubble caps the vapor-liquid phases are at the turbulent stats and perform intensive heat and mass exchange, and then the two phases are injected horizontally from the small holes oil the bubble cap wall. The vapor phase and the liquid drops are rolling around in the space between trays and then separate. The vapor phase rises to the upper level tray. Some of the small liquid drops injected from the bubble caps combines with other small drops and becomes a bigger one and fall on the tray with those big liquid drops. Some are absorbed into the bubble caps, bubbled and broken again, others flow into the next row of bubble caps with the liquid on the tray, or flow out round the bubble caps and to the lower level tray through the downcomer.

Referring to FIG. 3, the jet co-flow tray is the combination of the vertical sieve tray and the structured packing tray. Gas below the tray passes through the rising gas holes at a certain velocity. The liquid on the tray is extracted by the extract tube and the gap between trays. The gas and liquid pass through the upflow tube together, mix and exchange mass by intensive turbulence, then gas liquid flow into the feed materials to further strengthen the mass exchange, and finish the gas liquid separation. The gas rises to the upper tray by pressure difference. The liquid generally falls onto the tray as clear liquid, flows to the lower tray through the downcomer. This process engenders on every tray, the vapor-liquid is at overall countercurrent circumstances along the whole rectification section, the multi-stage co-flow operation is realized.

The guide valve tray (referring to FIG. 4) preserves the advantage of ordinary valve trays, such as high operation flexibility, good vapor-liquid contact conditions, high mass exchange efficiency; at the same time it overcomes the disadvantage such as large liquid surface gradient, large amount of liquid backmix on the tray, stagnant region in the arch area on the sides of the tray, the valve is easy to be worn out and removed, etc. Therefore, the guide valve tray is different from the regular valve tray in structure. First, there are guide holes with propriety size on the valve, the opening of the guide holes have the same direction of the liquid flow. Using the limited amount of gas flow from the guide valve to move the liquid can reduce the liquid surface gradient. Secondly, regular valves are in round shape, vapor liquid flow radially, it causes notable liquid backmixing which will reduce the efficient of the tray. The guide valve tray is in rectangular shape having valve legs on two sides. The gas flows from the two sides instead of radially, and perpendicularly to the liquid flux which tremendously decreases the backmixing. Thirdly, for regular bubble tray, in the arch area of the two sides of the tray liquid flows circularly, it's called liquid stagnant region. Because of the liquid stagnant region, gas in this area is like a side line, seldom has mass exchange, as a result the tray efficiency is reduced. Because the guide valve tray has one or two guide holes oil the valve, the proper arrangement of the valve can eliminate the stagnant region. Since the flow rout in the stagnant region is long, some of the valves in this region have two guide holes to speed the flow and equalize the liquid flux on the tray. Also, the structure of the guide valve is reliable, not easy to be worn out and removed, and is safe to operate. So the guide valve tray has the advantage of the regular valve tray, overcomes their disadvantage, has good fluid mechanics and mass transfer properties.

The present invention provides a method of (meth)acrylate production distilling (meth)acrylate from aqueous solution of (meth)acrylate which is embodied in such a manner: the number of ideal trays is at least 3, including rectification section and stripping section. The rectification section is using structured packing tray or jet co-flow tray, or using structured packing tray in upper section, and using jet co-flow tray in lower section. The number of jet co-flow tray and structured packing tray is in the ratio from 4:1 to 1:4, optimally from 3:2 to 2:3. The structured packing tray used in the upper section of the rectification section can be ripple ceramic packing, metal large-wave board, metal mesh packing, or ceramic structured packing. The stripping section is using guide valve tray or vertical sieve tray, or using guide valve tray in upper section and using vertical sieve tray in lower section. The number of guide valve trays and vertical sieve trays are in the ratio from 4:1 to 1:4, optimally from 3:2 to 2:3. The weir height of the jet co-flow tray and vertical sieve tray is 5˜20 mm. The operation temperature of the column bottom is 50˜90° C., optimally 70˜85° C. The pressure drop of the column is 2˜10 kPa, optimally 3˜8 kPa.

In a preferred embodiment of the present invention, the rectification section of the (meth)acrylate distillation column is using structured ripple ceramic packing tray, the stripping section is using vertical sieve tray. Comparing with column using same number of large holes sieve trays, under the same condition, the number of the ideal trays increases 150%. In another preferred embodiment, the upper section of the rectification section is still using structured ripple ceramic packing tray, the lower section of the rectification section is using jet co-flow packing tray. The stripping section is using guide valve tray in upper section and using vertical sieve tray in lower section. In this case, comparing with column using same number of large holes sieve trays, under the same condition, the number of the ideal trays increases 120%.

Considering the polymerization happens most likely in the stripping section, in the present invention, the stripping section is using guide valve tray in upper section and vertical sieve tray in lower section. This structure not only has relatively larger flux rate and higher tray efficiency, but also is easy clean. At the same time, increasing the height of the weir of the vertical sieve tray and jet co-flow packing tray properly will enhance the liquid thickness, encourage the liquid vapor contact, and improve the separation efficiency. Opening holes at the top of the mass transferring unit will lower the pressure drop inside the column, comparing to the large holes sieve tray, the column capability increase close to 50%, the (meth)acrylate content at the top of the column is decreased, raw materials consumption is reduced as well.

### Embodiment 1

The aqueous solution of acrylate separation column is using decompression distillation column. The height of the column is 20000 mm. The diameter of the column is 1500 mm. In this embodiment, the rectification section of the column is using ripple ceramic packing with a void fraction of 75%, the stripping section is using vertical sieve tray. The top of the mass transferring unit of the vertical sieve tray has open holes, to reduce the pressure drop. The vertical sieve tray of this embodiment can prevent the possible small amount of polymer to block the tray. Even if a small amount of polymer is produced, it is also easy to be cleaned. The height of the weir is 10 mm.

Continuously feed aqueous solution of acrylate containing 45% (mass) acrylate, 2% (mass) acetic acid, and small amount of formic acid and formaldehyde into aqueous solution of acrylate separation column, feed in the middle of the column, use toluene as azeotropic solvent for decompression azeotropic distillation, add hydroquinone and phenothiazine at the top of the column as inhibitor. During the operation, the pressure drop of the whole column is 4.5 kPa. The operation temperature of the bottom of the column is 78° C. Water mass content of the bottom after removing the water and most of the acrylate is less than 0.3%. The acetic acid mass content is less than 1.5%. The acrylate mass content in the ater phase at the top of the column is less than 0.30. The number of the ideal trays in the stripping section is 4. The number of the ideal trays (theoretic trays) in the rectification section is 10. The number of the ideal trays of the whole column is 14.

### Embodiment 2

The aqueous solution of acrylate separation column is using decompression distillation column. The height of the column is 20000 mm. The diameter of the column is 1500 mm. In this embodiment, the rectification section of the column is using ripple ceramic packing with a void fraction of 75%. The stripping section is using guide valve tray. In order to improve the liquid vapor mass transferring, the height of the weir of the guide valve tray is 10 mm. The guide valve tray of this embodiment can prevent the possible small amount of polymer to block the tray. Even if a small amount of polymer is produced, it is also easy to be cleaned.

Continuously feed aqueous solution of acrylate containing 45% (mass) acrylate, 2% (mass) acetic acid, and small amount of formic acid and formaldehyde into aqueous solution of acrylate separation column, feed in the middle of the column, use toluene as azeotropic solvent for decompression azeotropic distillation, add hydroquinone and phenothiazine at the top of the column as inhibitor. During the operation, the pressure drop of the whole column is 4.5 kPa. The operation temperature of the bottom of the column is 78° C. Water mass content of the bottom after removing the water and most of the acrylate is less than 0.3%. The acetic acid mass content is less than 1.5%. The acrylate mass content in the water phase at the top of the column is less than 0.3%. The number of the ideal trays in the stripping section is 6. The number of the ideal trays in the rectification section is 10. The number of the ideal trays of the whole column is 16.

### Embodiment 3

The aqueous solution of acrylate separation column is using decompression distillation column. The height of the column is 20000 mm. The diameter of the column is 1500 mm. In this embodiment, the upper section of the rectification section of the column is using ripple ceramic packing with a void fraction of 75%, the lower section is using jet co-flow packing tray, the stripping section is using vertical sieve tray. The top of the mass transferring unit of the vertical sieve tray has open holes, to reduce the pressure drop. In order to improve the liquid vapor mass transferring, the height of the weir of the guide valve tray is 15 mm. The jet co-flow packing tray of this embodiment can prevent the possible small amount of polymer to block the tray. Even if a small amount of polymer is produced, it is also easy to be cleaned.

Continuously feed aqueous solution of acrylate containing 45% (mass) acrylate, 2% (mass) acetic acid, and small amount of formic acid and formaldehyde into aqueous solution of acrylate separation column, feed in the middle of the column, use toluene as azeotropic solvent for decompression azeotropic distillation, add hydroquinone and phenothiazine at the top of the column as inhibitor. During the operation, the pressure drop of the whole column is 5.5 kPa. The operation temperature of the bottom of the column is 80° C. Water mass content of the bottom after removing the water and most of the acrylate is less than 0.5%. The acetic acid mass content is less than 2%. The acrylate mass content in the water phase at the top of the column is less than 0.6%. The number of the ideal trays in the stripping section is 4. The number of the ideal trays in the rectification section is 8. The number of the ideal trays of the whole column is 12.

### Embodiment 4

The aqueous solution of acrylate separation column is using decompression distillation column. The height of the column is 20000 mm. The diameter of the column is 1500 mm. In this embodiment, the upper section of the rectification section of the column is using ripple ceramic packing with a void fraction of 75%, the lower section is using jet co-flow packing tray, the stripping section is using guide valve tray. The top of the mass transferring unit of the vertical sieve tray has open holes, to reduce the pressure drop. In order to improve the liquid vapor mass transferring, the height of the weir of the guide valve tray is 15 mm. The jet co-flow packing tray of this embodiment can prevent the possible small amount of polymer to block the tray. Even if a small amount of polymer is produced, it is also easy to be cleaned.

Continuously feed aqueous Solution of acrylate containing 45% (mass) acrylate, 2% (mass) acetic acid, and small amount of formic acid and formaldehyde into aqueous solution of acrylate separation column, feed in the middle of the column, use toluene as azeotropic solvent for decompression azeotropic distillation, add hydroquinone and phenothiazine at the top of the column as inhibitor. During the operation, the pressure drop of the whole column is 5.5 kPa. The operation temperature of the bottom of the column is 80° C. Water mass content of the bottom after removing the water and most of the acrylate is less than 0.5%. The acetic acid mass content is less than 2%. The acrylate mass content in the water phase at the top of the column is less than 0.6%. The number of the ideal trays in the stripping section is 6. The number of the ideal trays in the rectification section is 8. The number of the ideal trays of the whole column is 14.

### Embodiment 5

The aqueous solution of acrylate separation column is using decompression distillation column. The height of the column is 20000 mm. The diameter of the column is 1500 mm. In this embodiment, the upper section of the rectification section of the column is using ripple ceramic packing with a void fraction of 75%, the lower section is using jet co-flow packing tray, the upper section of the stripping section is guide valve tray, the lower section is using vertical sieve tray. The top of the mass transferring unit of the vertical sieve tray has open holes, to reduce the pressure drop. In order to improve the liquid vapor mass transferring, the height of the weir of the guide valve tray is 15 mm. The jet co-flow packing tray, guide valve tray, and vertical sieve tray of this embodiment can prevent the possible small amount of polymer to block the tray. Even if a small amount of polymer is produced, it is also easy to be cleaned. The height of the weir is 10 mm.

Continuously feed aqueous solution of acrylate containing 45% (mass) acrylate, 2% (mass) acetic acid, and small amount of formic acid and formaldehyde into aqueous solution of acrylate separation column, feed in the middle of the column, use toluene as azeotropic solvent for decompression azeotropic distillation, add hydroquinone and phenothiazine at the top of the column as inhibitor. During the operation, the pressure drop of the whole column is 5 kPa. The operation temperature of the bottom of the column is 80° C. Water mass content of the bottom after removing the water and most of the acrylate is less than 0.4%. The acetic acid mass content is less than 1.8%. The acrylate mass content in the water phase at the top of the column is less than 0.5%. The number of the ideal trays in the stripping section is 5. The number of the ideal trays in the rectification section is 8. The number of the ideal trays of the whole column is 13.

### Comparison Embodiment 1

The aqueous solution of acrylate separation column is using decompression distillation column. The height of the column is 20000 mm. The diameter of the column is 1500 mm. In this embodiment, the rectification section and the stripping section are both using large holes perforated sieve trays with the perforated rate of 10˜20%.

Continuously feed aqueous solution of acrylate containing 45% (mass) acrylate, 2% (mass) acetic acid, and small amount of formic acid and formaldehyde into aqueous solution of acrylate separation column, feed in the middle of the column, use toluene as azeotropic solvent for decompression azeotropic distillation, add hydroquinone and phenothiazine at the top of the column as inhibitor. During the operation, if the acetic acid content in the bottom of the column is controlled lower than 2%, the pressure drop of the whole column will be 8 kPa. The consumption of acrylate at the top of the column will be larger than 2%. The operation temperature of the bottom of the column will be 88° C. or higher, which will be a big problem for stable operation. The number of the ideal trays in the stripping section and in the rectification section is both 2. The number of the ideal trays of the whole column is 4. Obviously, the traditional trays seriously constrain the expansion of the production of the aqueous solution of acrylate separation equipment, using new composite structure of the column is necessary.

### Comparison Embodiment 2

The aqueous solution of acrylate separation column is using decompression distillation column. The height of the column is 20000 mm. The diameter of the column is 1500 mm. In this embodiment, the upper section of the rectification section of the column is using jet co-flow packing tray, the lower section is using vertical sieve tray, the stripping section is using large holes perforated sieve trays with the perforated rate of 10˜20%.

Continuously feed aqueous solution of acrylate containing 45% (mass) acrylate, 2% (mass) acetic acid, and small amount of formic acid and formaldehyde into aqueous solution of acrylate separation column, feed in the middle of the column, use toluene as azeotropic solvent for decompression azeotropic distillation, add hydroquinone and phenothiazine at the top of the column as inhibitor. During the operation, the acetic acid content in the bottom of the column is controlled lower than 1.5%, the consumption of acrylate at the top of the column is lower than 1.0%. The pressure drop of the whole column is kept around 6.5 kPa. The operation temperature of the bottom of the column is maintained at 83° C. The number of ideal trays of the whole column is 7.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

1-15. (canceled)

16. A meth(acrylic) acid production system, comprising:
means for providing meth(acrylic) acid gas mixture by catalytic oxidation, wherein said gas mixture contains air, steam and a compound of one of C₃ compound and C₄ compound, wherein said C₃ compounds are selected from the group consisting of propane, propylene, isopropyl alcohol, glycerol or acrolein, wherein said C₄ compounds are selected from the group consisting of isobutane, isobutylene, tert-butanol or methylpropenal;
means for said gas mixture by water to form a solution mixture;
a distillation column having an upper rectification section, a lower stripping section, and a plurality of spaced apart trays supported in said rectification section and said stripping section, wherein the number of theoretic tray of said distillation column is at least 3, wherein impure meth(acrylic) acid from said solution mixture is separated in said distillation column that solution of said impure meth(acrylic) acid is collected at said stripping section of said distillation column; and
means for removing acetic acid from said impure meth(acrylic) acid, wherein meth(acrylic) acid is produced when said acetic acid is removed.

17. The meth(acrylic) acid production system, as recited in claim 16, wherein said trays at said rectification section of said distillation column are structured packing trays and jet co-flow packing trays, wherein said jet co-flow packing trays are positioned below said structured packing trays.

18. The meth(acrylic) acid production system, as recited in claim 17, wherein said trays at said stripping section of said distillation column are guide valve trays and vertical sieve trays, wherein said vertical sieve trays are positioned below said guide valve trays.

19. The meth(acrylic) acid production system, as recited in claim 18, wherein the number of said jet co-flow trays and said structured packing trays is in the ratio from 3:2 to 2:3, wherein the number of said guide valve trays and said vertical sieve trays is in the ratio from 3:2 to 2:3.

20. The meth(acrylic) acid production system, as recited in claim 19, wherein said structured packing tray provided in said rectification section is one of ripple ceramic packing tray, metal large-wave board, metal mesh packing tray, and ceramic structured packing tray.

21. A method of meth(acrylic) acid production, comprising the steps of:
(a) providing meth(acrylic) acid gas mixture by catalytic oxidation, wherein said gas mixture contains air, steam and a compound of one of C₃ compound and C₄ compound, wherein said C₃ compounds are selected from the group consisting of propane, propylene, isopropyl alcohol, glycerol or acrolein, wherein said C₄ compounds are selected from the group consisting of isobutane, isobutylene, tert-butanol or methylpropenal;
(b) absorbing said gas mixture by water to form a solution mixture of impure meth(acrylic) acid, wherein said mixture of impure meth(acrylic) acid comprises 40% to 60% methacrylate by weight, 35% to 55% water by weight, and 2% to 3% acetic acid by weight;
(c) introducing said solution mixture into a distillation column, wherein said distillation column has an upper rectification section, a lower stripping section, and a plurality of spaced apart trays supported in said rectification section and said stripping section, wherein the number of ideal trays of said distillation column is at least 3;
(d) separating said impure meth(acrylic) acid from said solution mixture in said distillation column that solution of said impure meth(acrylic) acid is collected at said stripping section of said distillation column; and
(e) removing acetic acid from said impure meth(acrylic) acid to produce meth(acrylic) acid.

22. The method, as recited in claim 21, wherein each of said trays at said rectification section of said distillation column is one of structured packing tray and jet co-flow packing tray.

23. The method, as recited in claim 21, wherein said trays at said rectification section of said distillation column are structured packing trays and jet co-flow packing trays, wherein said jet co-flow packing trays are positioned below said structured packing trays.

24. The method, as recited in claim 23, wherein the number of said jet co-flow trays and said structured packing trays is in the ratio from 4:1 to 1:4.

25. The method, as recited in claim 23, wherein the number of said jet co-flow trays and said structured packing trays is in the ratio from 3:2 to 2:3.

26. The method, as recited in claim 21, wherein each of said trays at said stripping section of said distillation column is one of guide valve tray and vertical sieve tray.

27. The method, as recited in claim 21, wherein said trays at said stripping section of said distillation column are a guide valve trays and a vertical sieve trays, wherein said vertical sieve trays are positioned below said guide valve trays.

28. The method, as recited in claim 23, wherein said trays at said stripping section of said distillation column are guide valve trays and vertical sieve trays, wherein said vertical sieve trays are positioned below said guide valve trays.

29. The method, as recited in claim 27, wherein the number of said guide valve trays and said vertical sieve trays is in the ratio from 4:1 to 1:4.

30. The method, as recited in claim 27, wherein the number of said guide valve trays and said vertical sieve trays is in the ratio from 3:2 to 2:3.

31. The method, as recited in claim 28, wherein the height of weir of each of said jet co-flow trays and said vertical sieve trays is 5 to 20 mm.

32. The method, as recited in claim 21, wherein an operation temperature of the bottom of said distillation column is 50 to 90° C., wherein a pressure drop of said distillation column is 2 to 10 kPa.

33. The method, as recited in claim 28, wherein an operation temperature of the bottom of said distillation column is 50 to 90° C., wherein a pressure drop of said distillation column is 2 to 10 kPa.

34. The method, as recited in claim 21, wherein an operation temperature of the bottom of said distillation column is 70 to 85° C., wherein a pressure drop of said distillation column is 3 to 8 kPa.

35. The method, as recited in claim 28, wherein an operation temperature of the bottom of said distillation column is 70 to 85° C., wherein a pressure drop of said distillation column is 3 to 8 kPa.
